(19) United States
(12) Patent Application Publication
(10) Pub. No.: US 2012/0226186 A1
(43) Pub. Date: Sep. 6, 2012

(54) METHOD FOR DETERMINING THE LEVEL OF ANALGESIA OF A SEDATED OR NARCOTIZED INDIVIDUAL

(76) Inventors: Jan H. Baars, Berlin (DE); Falk Von Dincklage, Berlin (DE); Benno Rehberg-Klug, Sergy (FR)

(21) Appl. No.: 13/508,098

(22) PCT Filed: Nov. 8, 2010
(86) PCT No.: PCT/EP2010/067065

§ 371 Date: May 4, 2012

Related U.S. Application Data
(60) Provisional application No. 61/258,929, filed on Nov. 6, 2009.

(30) Foreign Application Priority Data
Nov. 6, 2009 (DE) 10 2009 053 256.0

(51) Int. Cl. A61B 5/05; A61B 5/00; A61B 19/00

(52) U.S. Cl. 600/554; 600/557; 600/555

(57) ABSTRACT

The invention pertains to a method for determining the level of analgesia of a sedated individual, to the use of an apparatus for determining the level of analgesia of a sedated individual (so called “pain monitor”), as well as the use of an evoked pain-specific reflex response of a sedated individual for determining the level of analgesia of a sedated individual.

The invention refers to a method for determining the level of analgesia of a sedated or narcotized or anesthetized individual, an apparatus for determining the level of analgesia of a sedated individual (so called “pain monitor”) and its use, as well as the use of an evoked pain-specific reflex response of a sedated individual for determining the level of analgesia of a sedated individual.

Introduction

Sufficient analgesia is an essential component of every intensive medical therapy, because intensive care therapy often causes pain. Insufficient analgesia (i.e. too low a level of analgesia) may put the patient into peril through agitation and stress reactions, whereas an overdose of analgesic therapy may cause an increase of the duration of artificial respiration.

Both cases put the patient at risk and lead to an increase in therapy costs. Therefore, it is standard practice to monitor analgesia. When the patient is awake, this may be done based on the self-assessment of the patient, even though this does not allow any conclusions about the pain that might be suffered during future medical procedures. For analgo-sedated patients, the assessment needs to be performed by third parties. Thus far, no apparatus-based procedure is known that allows for an objective measurement of the level of analgesia of a sedated individual.

The reflex threshold of nociceptive flexor reflexes corresponds to the subjective pain threshold of healthy individuals (Willer, 1977) and is changed in a concentration dependent manner through the application of analgetics like, for example, morphine (Willer, 1985).

The presently described “pain monitor” automatically determines the threshold of a nociceptive reflex using an algorithm. Therefore, according to the invention, an automated measurement of the threshold of a pain-specific reflex, like the blink reflex of the eye or the flexor reflex, is possible.

Studies are known in the state of the art that investigate the influence of anaesthetics on the RIII-reflex, a widely used flexor reflex of the lower limb. They show that the reflex response disappears after single stimulations at relatively low concentrations of the anaesthetics (Petersen-Felix et al., 1996). In this study, high dosages were used in which also motor reactions to painful stimulation were suppressed.

These results led to the conclusion that sedatives in a dosage that render an individual unconscious are not compatible with the conduction of pain-specific reflexes like the RIII-reflex (Petersen-Felix et al., 1995). Propofol, which is commonly used for sedating patients in intensive care units, was therefore used at very low dosages at which the patient were still awake and could participate in a reaction test (Petersen-Felix et al., 1996).

Accordingly, a prejudice existed in the state of the art that has been overcome with the present invention. The inventors have surprisingly found that a stimulation signal, like for example the reflex threshold of nociceptive reflexes, is suitable for an objective measurement of the analgesia under narcosis (sedation).

Description

In a first aspect, the invention pertains to the use of an apparatus for determining the reflex response of a conscious patient. Such an apparatus comprises at least the following components:
1. A stimulation unit for generating a stimulation signal. This stimulation signal is for triggering a pain-specific reflex in an individual.
2. A measurement unit for recording a pain-specific reflex response to the stimulation signal.
3. A control unit for adapting the intensity of the stimulation signal in case the evoked pain-specific reflex response does not occur or is too high. In that,
   the control unit decreases the intensity of the stimulation signal if the pain-specific reflex response caused by the stimulation signal is greater that a predetermined index value and
   increases the intensity of the stimulation signal if the pain-specific reflex response caused by the stimulation signal is below a predetermined index value.

Such an apparatus is described in von Dincklage, 2009 and schematically shown in FIG. 6.

According to the invention, such an apparatus is used for determining the level of analgesia of a sedated or narcotized individual. In other words, a value is generated that may serve a physician for deciding whether the sedated individual feels pain or not.

In a preferred embodiment, the control unit analyzes the recorded reflex response and checks for artefacts to correct for erroneous measurements. This is performed using algorithms known to a person of skill in the art. The preferred embodiment uses electrode positions for stimulation and recording as shown in FIG. 7 yielding signals shown in FIG. 8. An example of the adjustment of the stimulation intensity by the control unit is given in FIG. 9. The calculated reflex threshold may be determined by means of logistic regression as shown in FIG. 10.

In a second aspect, the invention refers to a method for determining or estimating the level of analgesia of a sedated individual. This method comprises the following steps:
1. Firstly, a stimulation signal for triggering as pain-specific reflex in an individual is generated.
2. Subsequently, the magnitude of the triggered pain-specific reflex is measured. When the reflex value does not correspond to a predetermined index value, the intensity of the stimulation signal that causes the reflex is adapted using a feedback loop which is in one embodiment of the invention computer-controlled. The feedback loop allows for the determination of a calculated stimulation current for triggering a reflex of a predetermined index value. The algorithm of the threshold determination depends preferably on a bidirectional step model with a variable step width, preferably in combination with a logistic regression to estimate the threshold from the last 4 to 40 stimuli. The variable step width depends on the stability of the underlying individual threshold value.
3. The stimulation frequency preferably varies between 3-20 stimuli per minute. Higher stimulation frequencies allow a faster determination of the threshold underlying individual threshold. When the measured value of the triggered pain-specific reflex deviates from the index value, an adaptation of the intensity of the stimulation signal occurs.
4. The calculated stimulation necessary to generate a reflex of the index value is determined by a logistic regression of the 4-40 stimuli. This determination may be repeated after every stimulation (sequence).
5. This calculated stimulation intensity is directly dependent on the level of analgesia. The calculated stimulation intensity value may be displayed, indicating the level of analgesia.

The stimulation signal (a noxious stimulus) can be an electrical, a mechanical or a thermal signal. It is preferred that the signal is an electrical signal.

In a third aspect, the invention refers to the use of an evoked pain-specific reflex response of a sedated individual for determining the level of analgesia of the sedated individual. This aspect is based on the finding that the reflex threshold of an analgo-sedated patient allows the prediction of pain reaction in response to a painful procedure.

The pain-specific reflex used can be chosen from the group of protective reflexes, like the blink reflex of the eye and the flexor reflexes, in particular of the lower extremities.

Preferably, the analgo-sedation is performed using a sedative (sleeping medicine) that is chosen from a group consisting of propofol(2,6-diisopropylphenol), benzodiazepines and alpha-2-antagonists. The analgetics may be chosen from the group consisting of opioids like, for example, morphine, sufentanil, remifentanil or fentanyl.

The stimulation signal can consist of one stimulus or a sequence of stimuli (at least two), in particular of similar amplitude. When the stimulation signal is electrical, 1 to 5 single stimuli with a duration of 0.5 ms to 2 ms each with a frequency of 150 Hz to 250 Hz, in particular of 200 Hz, are preferably applied. The stimulation signals can be applied with pauses of up to 20 s.

The maximum energy that is to be applied to the individual with the stimulus impulse or the sequence of stimuli is 0.3 to 0.8 Joule, preferable 0.5 Joule. The maximum voltage is 700 V, preferred are 600 V.

The reflex signal triggered by the stimulation signal is registered within a time period of 1 ms to 1 s after the stimulation. Depending on the magnitude of this registered signal, the stimulation current of the following impulse is adapted such that the stimulation intensity is increased when the reflex response is below a predefined index value and is decreased when the reflex response is higher than the predefined index value, such that the calculated stimulation intensity is reached so that a reflex with the magnitude of the index value is triggered.

The index value of the reflex response may be between the reflex threshold and the highest possible amplitude, which depends on the nerve used and the characteristics of the individual. In a preferred embodiment of the invention, the index value is the threshold value for triggering the respective reflex. The threshold value is usually between 5 to 15 times the standard deviation of the electrical base signal recorded from the target muscle.

The calculated stimulation current is a parameter of the pain sensitivity of the individual. This parameter might help a physician to decide whether the drug concentration used for analgesia of the patient is appropriate or should be adapted by applying more or less of the analgesic. In other words, the calculated stimulation current is a measurement value that may form the basis for the decision of a physician.

### FIGURES

This invention is now further described with reference to the figures.

FIG. 1: Drug administration protocol: constant propofol concentration plus variable concentrations of remifentanil. LOC=loss of consciousness

For this drug administration protocol, the plasma concentration (Ce) of propofol was increased in steps of 1 mg/l every 15 min until loss of consciousness, defined as no reaction to verbal stimuli. Subsequently, the propofol plasma concentration was decreased by 1 mg/l and after 15 min of equilibration, additional administration of remifentanil was started. The remifentanil plasma concentration was increased in steps of 0.5 pg/l every 10 min up to 3 steps above the loss of reaction to pain stimuli. After the maximal remifentanil concentration was kept constant for 10 min, the administration of remifentanil was stopped and after additional 30 min, the administration of propofol was stopped. In order to shorten the time until the concentration equilibrium between the plasma and the effector organs brain and spinal cord were reached, higher concentration than the target plasma concentration of both medicaments were reached for a short time. The measurement of the reflex threshold was performed after reaching equilibrium between plasma compartment and effector compartment.

FIG. 2: Drug administration protocol 2: High propofol concentration plus a low remifentanil concentration versus low propofol concentration plus high remifentanil concentration. LOC=loss of consciousness

For this drug administration protocol, the plasma concentration of propofol was increased in steps of 1 mg/l each 15 min until loss of consciousness, defined as no reaction to verbal stimuli. Subsequently, the plasma concentration of remifentanil was increased in steps of 0.5 pg/l each 10 min until loss of reaction to pain stimuli. After 10 min of equilibration, propofol plasma concentration was decreased in steps of 1 mg/l each 15 min until reactions to pain stimuli reoccurred. After additional 15 min for equilibration, the remifentanil plasma concentration was increased in further steps of 1 pg/l every 10 min until reactions to pain stimuli again subsided. Then, the administration of propofol was stopped and after reactions to the pain stimuli reoccurred, the administration of remifentanil was stopped. In order to reach concentration equilibrium faster between plasma and the effector organs brain and spinal cord, concentrations of both medicaments were reached for short times that were higher than the target plasma concentrations. The measurement of the reflex threshold was performed after reaching equilibrium between plasma compartment and effector compartment.

FIG. 3: Shown are the individual data points for the BIS and the RIII reflex threshold after the loss of consciousness for the first drug delivery protocol. For each sequence of the reaction test (verbal instructions, squeezing of the Musculus trapezius, 30 s tetanic stimulus at 80 mA), the last RIII reflex threshold value and the last BIS value (which were taken immediately before the beginning of test sequence and are therefore not influenced by the test sequence) were included. From the data shown, the following prediction probabilities were calculated (value±standard deviation): RIII reflex threshold value: 0.86±0.02 and BIS 0.84±0.02.

FIG. 4: Population prediction probability for drug administration protocol 2

Shown are the individual data points for the BIS and the RIII reflex threshold after the loss of consciousness for the second drug administration protocol. For each sequence of the reaction test, the last RIII reflex threshold value and the last BIS value (which were obtained directly before the beginning of the test sequence and were therefore not influenced by the test sequence) were included. From the data shown, the following prediction probabilities were calculated (value±standard deviation): RIII reflex threshold: 0.77±0.04 and BIS 0.64±0.05.

FIG. 5: Concentration response Curves

The curves show the increase of the pain reflex threshold of single individuals which increasing concentrations of remifentanil and constant concentrations of propofol according to protocol 1. The propofol concentration was titrated individually such that after loss of consciousness, it was reduce by 1 μg/ml and then held constant (2 μg/ml to 4 μg/ml plasma concentration). Normalization of the reflex threshold was performed with regard to the threshold at the loss of consciousness. The concentrations of hypnotics and analgesics were in the range of those used in intensive medicine for analgosedation.

FIG. 6: Schematical depiction of an apparatus 1 for determining the level of analgesia of a sedated individual 8 including the relationships of its components.

The apparatus 1 comprises in the embodiment shown the following components:
a stimulation unit 2 for generating a stimulation signal 7 with the aim of triggering a pain-specific reflex in an individual 8,
a measurement unit 3 for recording a pain-specific reflex response signal to the stimulation signal 7, and
a control unit 4 for adapting the intensity of the stimulation signal 7.

First, an index value 5 of the reflex response signal that is to be generated is stored in the control unit 4, which may be a computer. This index value of the reflex response signal may be equivalent, for example, to the reflex threshold of the individual.

The control unit 4 gives a control signal 6 to the stimulation unit 2, which then generates at least one stimulation signal 7 for triggering a pain-specific reflex. This stimulation signal 7 is preferably an electrical signal, but can also be a thermal or tactile/mechanical signal. This stimulation signal 7 is transferred to a sedated individual 8 such that the pain-conducting nerve fibers of the individual 8 are stimulated with a purpose of triggering a pain-specific reflex. The choice of area/location of the stimulation determines the respective muscle for recording the contraction. For example, when choosing the outer side of the ankle for stimulation, the recording is performed at a thigh muscle that will be activated in response to the stimulation.

Shortly after releasing of the stimulation signal 7 through the stimulation unit 2, the measurement unit 3 expects and measures a reflex response signal, e.g. in the form of an electromyogram (EMG) signal (reflex response) 9 above the contracting muscle.

For this purpose, the measurement unit 3 may comprise an analogue to digital converter (ADC) and a bio-signal amplifier. When the intensity of the reflex response signal is not equal to the index value 5 of the reflex response, the control unit 4 adapts the single intensity of the stimulation signal 7 as follows: the intensity of the stimulation signal 7 is decreased by the control unit 4 when the pain-specific reflex response signal triggered by the stimulation signal 7 is higher than the index value 5 of the reflex response. When the pain-specific reflex response signal triggered by the stimulation signal 7 is below the index value 5 of the reflex response, the control unit 4 increases the intensity of the stimulation signal 7 that is to be released next. When the intensity of the reflex response signal is equal to the index value 5 of the reflex response, the control unit 4 does not adapt the signal intensity of the stimulation signal 7.

The adaptation of the intensity of the stimulation signal that stimulates the reflex is performed using the control signal 6 from the control unit 4, preferably using a feedback loop for determining the value/amplitude of a calculated stimulation signal 9, i.e. of a calculated stimulation current or a calculated stimulation intensity. This calculated signal intensity 9 will trigger a reflex response signal of the predetermined index value 5 of the reflex response signal.

In the measurement unit 3, the signals that were registered through EMG-electrodes over the muscle are amplified, filtered and digitalized.

Preferably, the calculated stimulation intensity 9 is determined from several reflex response signals measured by the measurement unit 3 and its respective stimulation intensities, where the calculated stimulation intensity 9 is necessary for reaching the index value 5 of the reflex response (e.g. of the reflex threshold). This calculation may be updated after each stimulation and preferably performed using logistic regression in the measurement unit 4 of the apparatus 1. The calculated stimulation intensity 9 that is determined from the last (e.g. ten) recorded stimulations is preferably shown as a numerical value on a display means 11, e.g. on a screen or display. On the basis of this value, a physician can decide whether the depth of the analgesia of the individual is appropriate or not. If a painful medical procedure is imminent, like a surgery, suctioning the trachea, etc., the depth of the analgesia can be adapted according to these needs.

FIGS. 7 to 10 describe a preferred embodiment of the present invention. The preferred embodiment uses electrode positions for stimulation and recording as shown in FIG. 7 yielding signals shown in FIG. 8. An example of the adjustment of the stimulation intensity by the control unit is given in FIG. 9. The calculated reflex threshold may be determined by means of logistic regression as shown in FIG. 10.

FIG. 7 shows the lower body part of a sedated individual whose sedation level (pain level) is to be determined. The individual is stimulated with an electrical stimulation signal from the stimulation unit of a pain monitor at the outer side of the ankle's skin using electrodes. A pain-specific reflex signal (EMG recording) that is generated by the stimulation signal is recorded with a measurement unit of the pain monitor.

FIG. 8 depicts the variation in intensity of the stimulation signal and the recorded pain-specific reflex signal that was measured by a measurement unit of the pain monitor 90 ms to 150 ms after the launch of the stimulation signal. A stimulation artefact shown on the left side of the EMG recording are filtered out by the control unit of the pain monitor.

The control unit decreases the intensity of the stimulation signal when the pain-specific reflex signal that is caused by the stimulation signal is above an index value of the reflex response, and the control unit increases the intensity of the stimulation signal when the pain specific reflex response caused by the stimulation signal is below a predetermined index value of the reflex response. In the shown case, a stimulation intensity of 7 mA, 8 mA, and 9 mA were launched and the pain-specific reflex signal was recorded (μV over ms).

FIG. 9 shows a graph of the measured stimulation intensity in mA over the time in seconds. Reflex responses above the threshold (triggering a muscular response) are shown in light squares, reflex responses below the threshold are shown in dark circles.

FIG. 10 shows, how the calculated reflex threshold is calculated. 12 successive stimulations are fitted with a logistic regression function: P=1/(1+exp(−(x−x0)/b)) with x0 the calculated stimulation intensity of 50% chance of reflex occurrence (reflex threshold) and b the steepness of the curve. P is the measured probability of reflex occurrence for a given stimulation intensity x. Given the 12 datapoints the reflex calculated threshold yields a value of 8.18 mA.

### EXAMPLE

Attempts to measure the level of analgesia in deeply sedated and therefore not communicating patients using scoring systems have revealed only insufficient information. Therefore, apparatus based monitoring methods were investigated regarding their suitability for determining the depth of sedation of the pain level. Such monitors exclusively use different approaches for interpretation of the EEG or of acoustic evoked potentials (AEP). The EEG monitor most used is the BIS-monitor. This monitor by Aspect Medical Systems, Inc. (Norwood, Mass., USA) is used since 1992 for measuring the dept of sedation. Other monitors that are based on EEG or acoustic evoked potentials are (as for example the BIS®-monitor) only used for monitoring the sedation, but not of the analgesia of a patient.

With the present invention, the inventors use an evoked reflex response to a short electrical stimulus that is well tolerated by a healthy individual. Using a computer based feedback loop, the intensity with which the reflex is stimulated is adapted such that it is always in the range of the reflex threshold. This method can be used with different pain specific reflexes, such as the flexor reflexes of the lower extremities or the eye blink reflex.

The threshold of the flexor reflexes corresponds in healthy individuals to the subjective pain threshold and reflex amplitude in healthy awake individuals changes in a concentration-dependent fashion to the administration of analgesics, like morphine. This connection between subjective pain sensation and the flexor reflex threshold permits its use as an objective pain parameter in the pharmacological and physiological pain research.

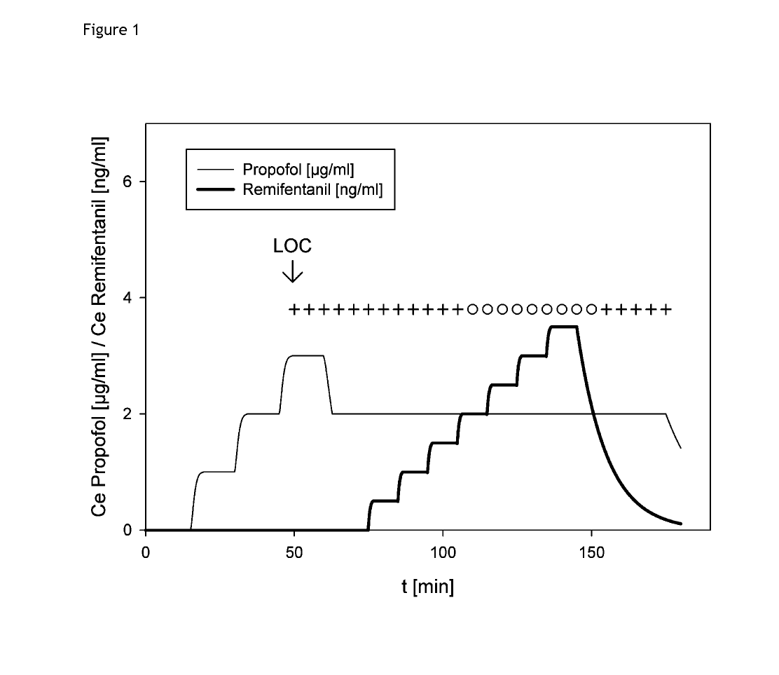

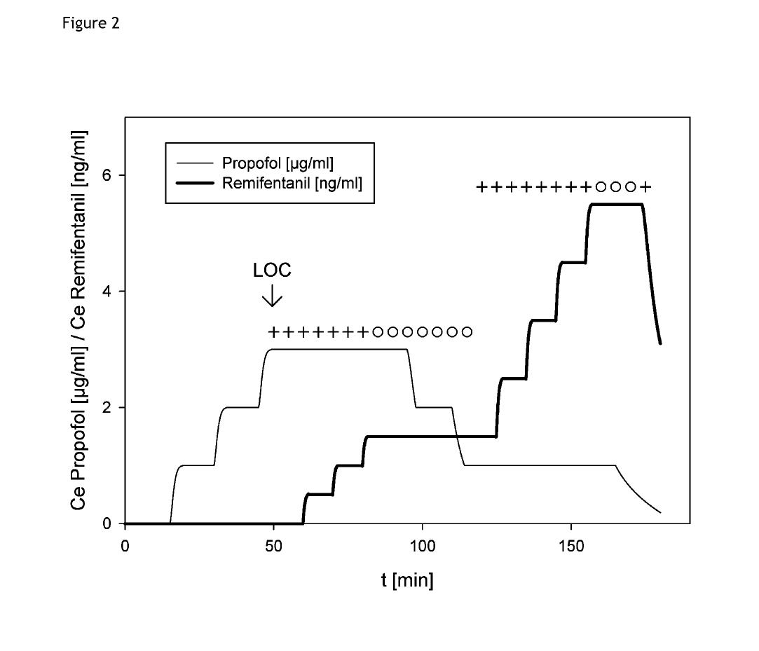

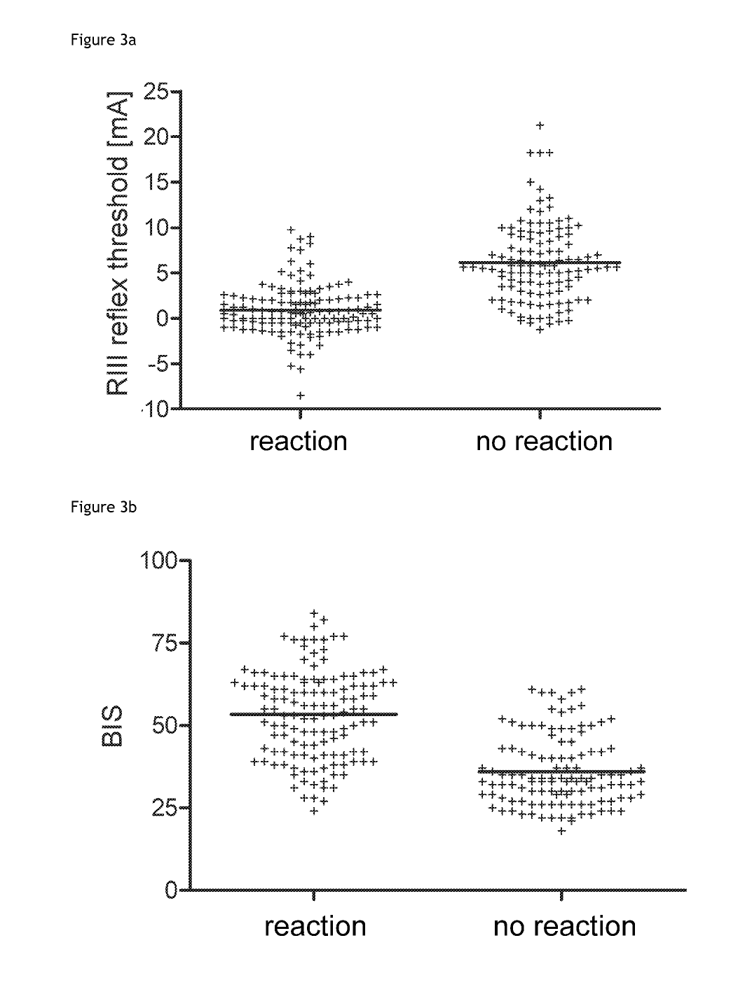

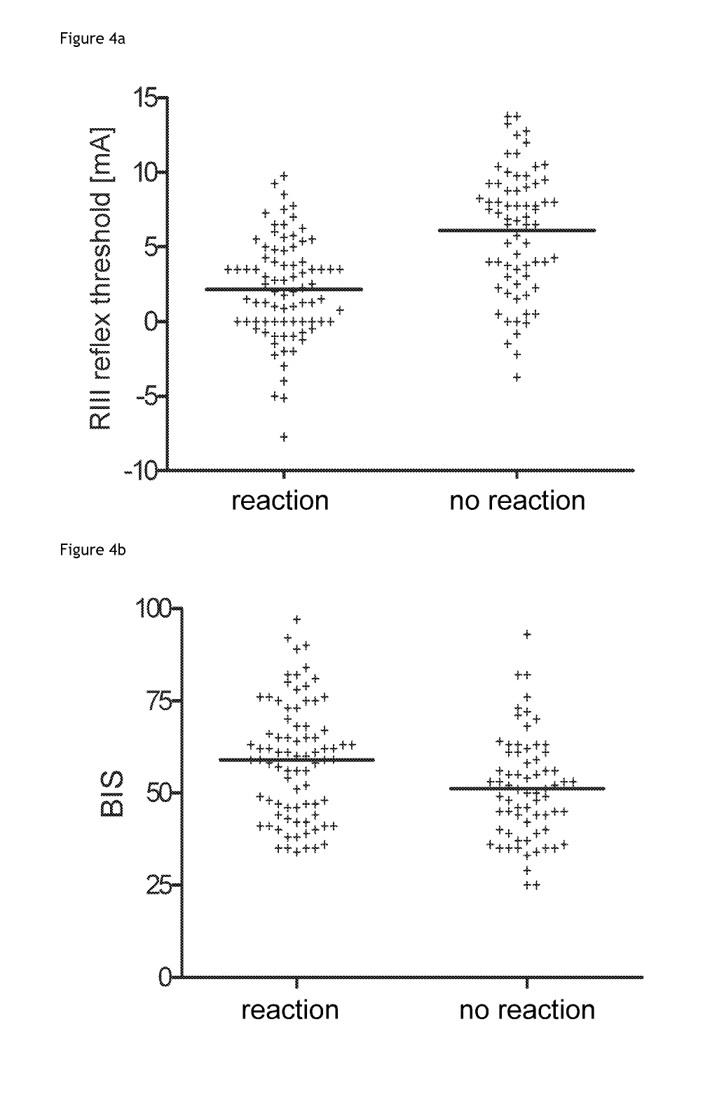

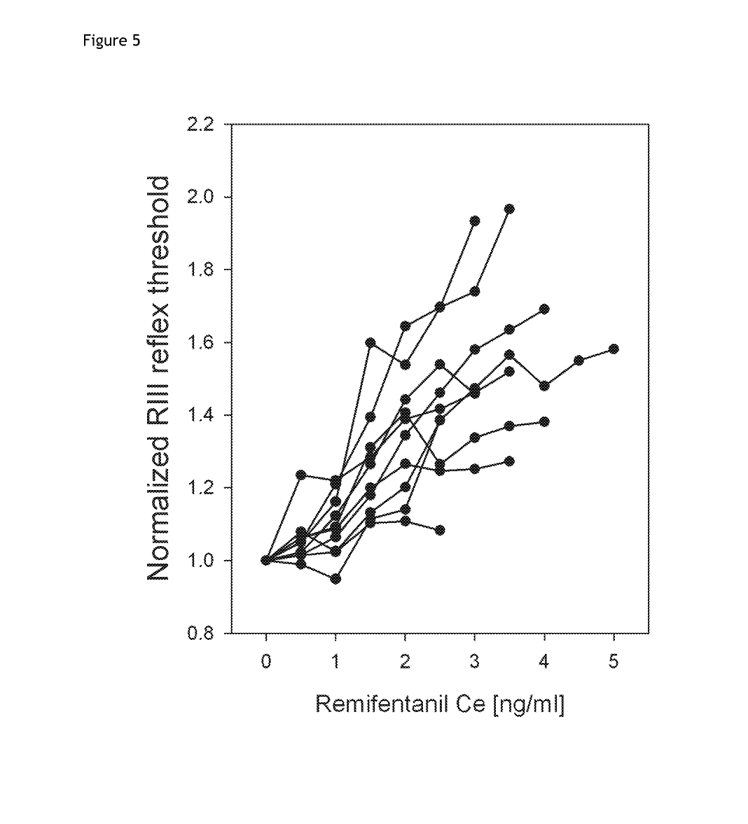

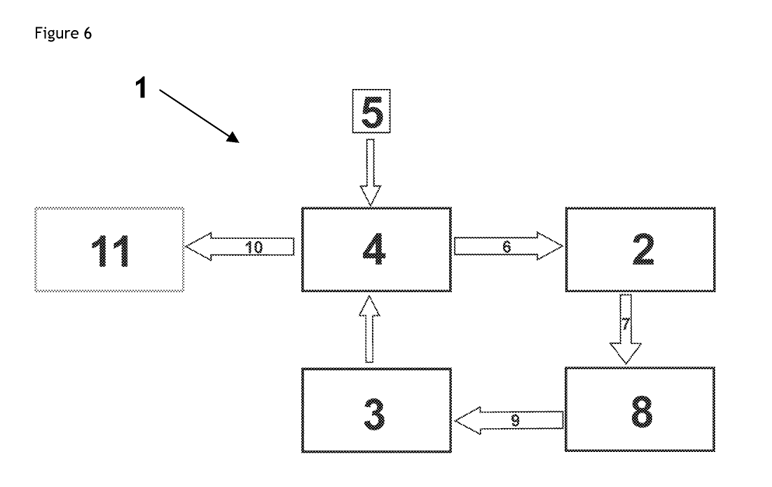

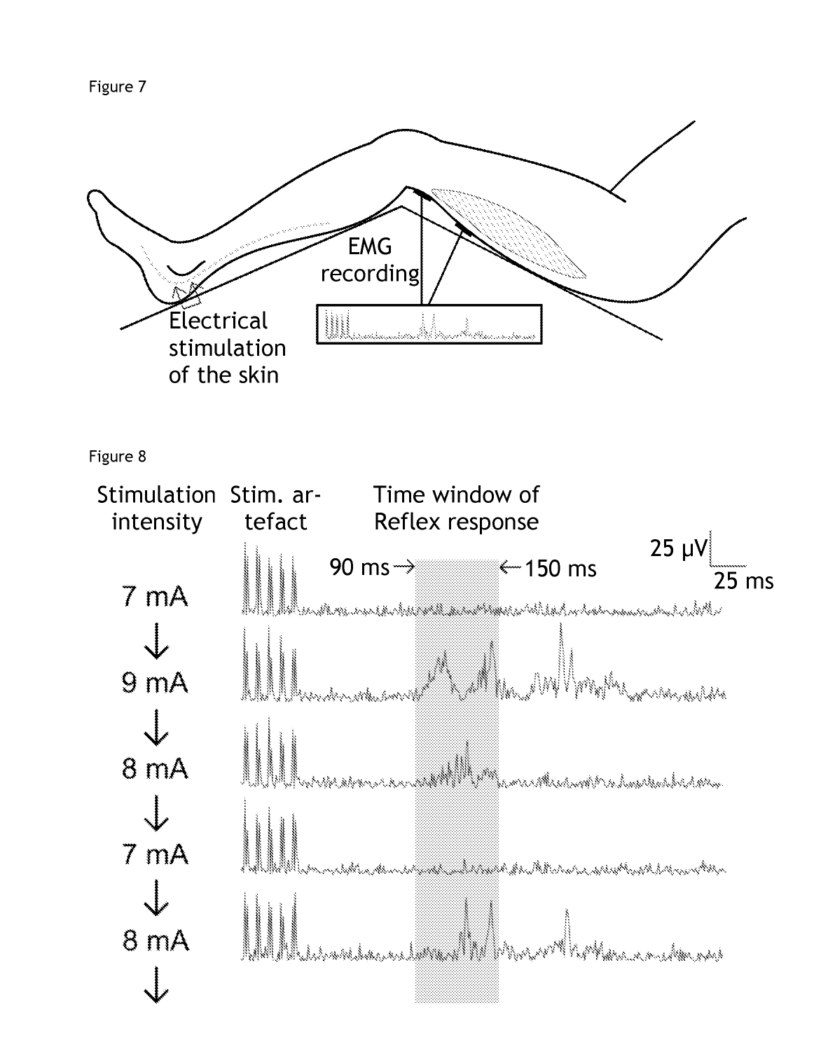

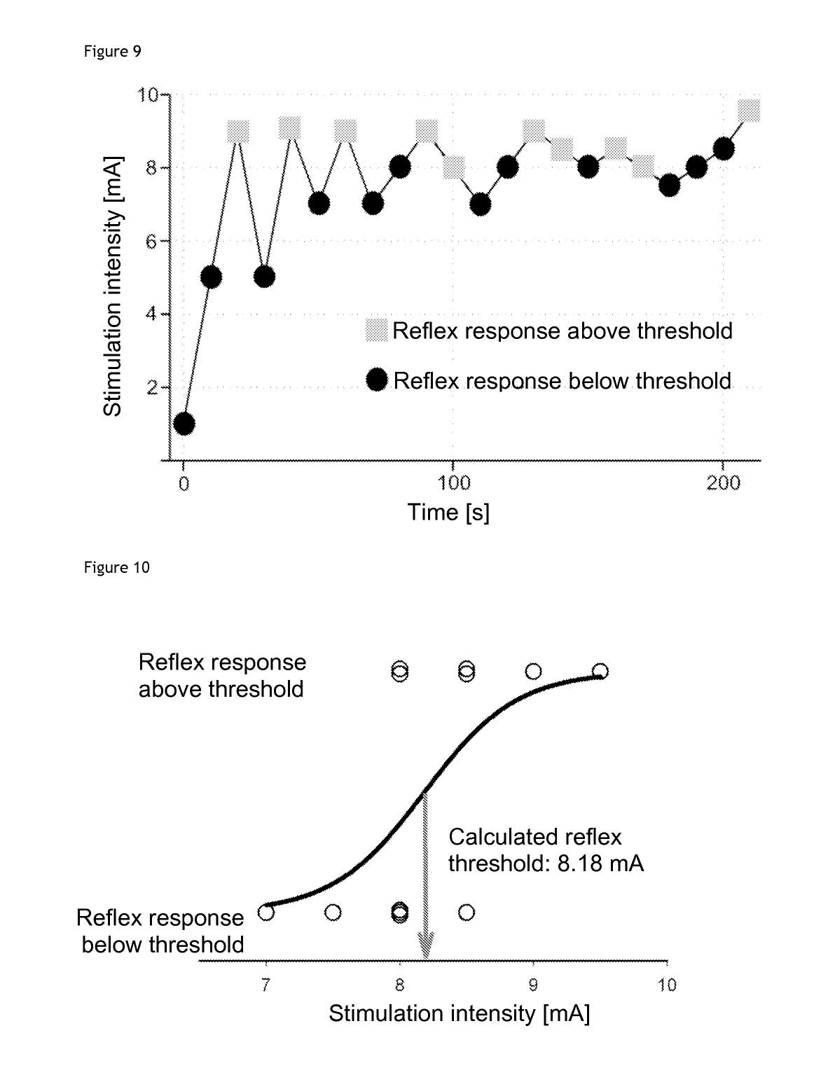

The sedative propofol that is used in intensive medicine may lead to a concentration depend change of the RIII reflex threshold. The inventor's investigations have shown (see FIGS. 1 to 5) that the flexor reflex threshold increases with increased administration of an opioide (remifentanil) even during the administration of propofol. Further, it could be shown that the RIII reflex threshold could be used to predict motor responses to pain stimuli better than parameters of the processed electroencephalogram (EEG).

Depth of anaesthesia can be defined as the probability of the lack of a response to a stimulus (Shafer et al 2008). Remaining motor reactions to pain stimuli are a sign for insufficient analgesia.

The RIII reflex as a component of the nociceptive flexor reflex (NFR) is a polysynaptic spinal reflex that is triggered through the stimulation of nociceptive afferent nerves. In order to measure the RIII reflex, the activity of the biceps femoris is measured with an electromyogram (EMG) during the application of electrical stimuli to the skin, such that the ipsilateral Nervus suralis is stimulated. On the basis of the measured EMG response, the intensity necessary for triggering the RIII reflex can be measured as an objective value for the individual nociceptive threshold (Sandrini, 2005; Skljarevski, 2002).

The inventors compared the RIII reflex threshold with the BIS (bispectral EEG index) value as a parameter for predicting movement responses to a painful electrical stimulus to a part of the skin. For this purpose, the prediction probabilities for both parameters were calculated while one of two different drug administration protocols was followed: In one drug administration protocol, the plasma concentration of propofol was held constant and only the concentration of remifentanil was increased in a stepwise manner in order to observe changes of the parameters to remifentanil(3-{4-methoxycarbonyl-4-[(1-oxopropyl)phenylamino]-1-piperidin}propionic acid-methyl ester) in clinical relevant concentrations until consciousness was reached. In the second drug administration protocol, different combinations of high propofol concentrations together with lower remifentanil concentrations as well as low propofol concentrations with high remifentanil concentrations were administered. The purpose of this second drug administration protocol was to differentiate whether the two drugs change the measured parameters in a similar manner.

Methods

Subjects and Setting

After approval of the local ethics committee (Berlin, Germany) and obtaining written informed consent, the study was performed in 20 healthy (ASA class I) male volunteers, ranging in age from 23 to 35 yr. Only male volunteers were included to reduce the variability of the RIII reflex threshold. During the course of the study, the subjects were comfortably rested in therapy beds with a flexed leg-section to maintain angles of 120° in the hip and 130° in the knee.

Automated RIII Threshold Tracking

The subjects were comfortably rested in therapy beds with a flexed leg-section to maintain angles of 120° in the hip and 130° in the knee. To elicit the RIII reflex of the left biceps femoris muscle, the left sural nerve was repeatedly stimulated at its retromalleolar pathway via surface electrodes (inter-electrode distance: 30 mm). Stimuli were applied automatically at randomized intervals of 8-12 s to avoid habituation, with each stimulus consisting of a volley of five rectangular electrical pulses of 1 ms duration each, at 200 Hz (DS5, Digitimer Ltd, Hertfordshire, UK). To record the RIII reflex of the left biceps femoris muscle, surface electrodes were placed over its lateral tendon and over the muscle itself, 10 cm proximal of the popliteal fossa. The recorded signals were amplified (g.BSamp, g.tec, Schiedlberg, Austria), digitized at a sampling rate of 5 kHz (Mikro 1401 mk II, CED Ltd, Cambridge, UK), rectified, and analysed using Signal 3.10 (CED Ltd).

In this study, the RIII reflex threshold was traced continually by an automated RIII threshold tracking system. This system varies the stimulus intensity according to an up-down-staircase algorithm with a variable step length to estimate the stimulus intensity associated with a 50% probability of RIII reflex occurrence, which is defined as the reflex threshold (von Dincklage et al. 2009). This is a commonly used standard model of RIII reflex threshold estimation, but here other than in single trial experiments, estimation of the threshold was not performed by calculating the midrun estimates of the up-down runs but by using a logistic regression of the last 12 reflexes recorded, to allow for a continual estimation of the threshold after every stimulus. RIII reflex occurrence was defined as an interval peak z score higher than 10.32 in the post-stimulation interval of 90-150 ms. (Rhudy et al 2007)

Testing Procedure for Reactions on Verbal and Noxious Stimuli

During the whole course of the study, the reactions on verbal and noxious stimuli were tested every 5 min. The testing sequence was performed in the following order: one single verbal command, loudly repeated verbal commands, trapezius squeeze of 10 s duration, electrical tetanic stimulation in the area of the right ulnar nerve with 80 mA for 30 s. The different stimuli were applied immediately one after another, with a maximum of 5 s in between. Any verbal or movement reaction, regardless of purposeful or not, was considered as a positive response and the sequence of reaction testing was aborted.

Drug Administration and Monitoring

The subjects fasted at least 6 h before the administration of the drugs. Before the study period, standard monitoring including non-invasive arterial pressure, electrocardiography, pulse oximetry, a tight-fitting face-mask for measuring end-tidal CO2, surface electrodes for the BIS, and an i.v. access via a forearm vein were established. To avoid hypoventilation and to maintain a stable level of end-tidal CO2 under higher drug concentrations, some subjects received Guedel tubes, assisted ventilation through the face mask, or both. Propofol and remifentanil were infused i.v. via computer-controlled infusion pumps, which were programmed with the weight- and age-corrected pharmacokinetic parameter set of Schnider and colleagues (Schider et al 1998) for propofol and with the body mass- and age-corrected pharmacokinetic parameter set of Minto for remifentanil(Minto et al 1997). The dosing of the drugs followed two different drug administration protocols, which were each applied to 10 of the subjects.

In the first administration protocol (FIG. 1), the effect compartment concentration (Ce) of propofol was increased in steps of 1 μg ml-1 every 15 min until the loss of consciousness, defined as the loss of reaction to verbal stimuli. Then propofol Ce was decreased by 1 μg ml-1 and after 15 min, to allow for equilibration processes, the additional administration of remifentanil was started. Remifentanil Ce was increased in steps of 0.5 ng ml-1 every 10 min up to three steps above the loss of reactions to the noxious stimuli. After the maximum remifentanil concentration was maintained for 10 min, the administration of remifentanil was discontinued and after another 30 min, the administration of propofol was discontinued.

In the second drug administration protocol (FIG. 2), the Ce of propofol was increased in steps of 1 μg ml-1 every 15 min until the loss of consciousness, defined as the loss of reaction to verbal stimuli. Then remifentanil Ce was increased in steps of 0.5 ng ml-1 every 10 min until the loss of reaction to the noxious stimuli. After 10 min time to allow for equilibration processes, propofol Ce was decreased in steps of 1 μg ml-1 every 15 min until reactions to the noxious stimuli reoccurred. After another 15 min for equilibration processes, remifentanil Ce was increased further in steps of 1 ng ml-1 every 10 min until reactions to the noxious stimuli ceased again. Then the administration of propofol was discontinued first. After reoccurrence of the reactions to the noxious stimuli, the administration of remifentanil was discontinued as well.

Data Analysis and Statistical Analysis

Analysis was performed with RIII reflex threshold and BIS data points obtained after the loss of consciousness. For each sequence of reaction testing as described above, the last RIII reflex threshold value and the last BIS value that were obtained immediately before starting the sequence of testing for a reaction and which therefore were not influenced by the testing sequence were used for analysis.

To compare the performance of the RIII reflex threshold with that of the BIS in distinguishing between reactions and the absence of reactions on the stimuli in each individual, the prediction probability PK was calculated for each method for every individual subject. A PK value of 1 stands for a 100% correct differentiation between reactions and the absence of reactions, whereas a value of 0.5 represents only a 50:50 chance of a correct differentiation. The estimation of individual PK-values was performed using the spreadsheet macro PKMACRO as described by Smith and colleagues (Smith et al 1996). Standard errors of the estimates were computed by the jackknife method. Individual PK-values for the RIII threshold and the BIS were compared for each drug administration protocol using a Wilcoxon signed-rank test.

To adjust for the large inter-individual variance of the RIII reflex threshold, the individual reflex threshold values were normalized to the first threshold which was estimated after the subject's loss of consciousness. This mode of normalization has been chosen to avoid the necessity of recording the RIII reflex in wake subjects and therefore to reduce the inconvenience of the procedure when the method would be used on patients.

To compare the overall performance of the RIII reflex threshold and the BIS in distinguishing between reactions and the absence of reactions on the noxious stimuli in all subjects, the population prediction probability PK was calculated for the BIS, the normalized RIII reflex threshold, and remifentanil effect compartment concentrations. However, the PK statistic used here is based on the assumption of independent data, since no comparable statistic method has been developed that permits non-independent data. Therefore, we used the PK statistic still while the assumption of independent data was violated for our data by the inclusion of multiple stimuli for each subject, as it has been done in other investigations (Hung et al. 1992, Katoh et al.1998, Leslie et al.1996, Rehberg et al. 2004). As a result of this, standard errors may have been underestimated in our analysis. Statistical testing of the prediction probabilities was performed using the spreadsheet macro PKDMACRO as described by Smith and colleagues (Smith et al. 1996).

Results

No relevant changes in arterial pressure, heart rate, arterial oxygen saturation, or end-tidal CO2 were observed throughout the study. The loss of reaction to repeated verbal commands for all 20 subjects occurred at a median effect compartment concentration of 4 μg ml-1 (range: 2-5 μg ml-1) propofol.

The median stimulus intensity that was applied through the automated threshold tracking system after the loss of consciousness of the subjects was 23.75 mA (min: 7.75 mA, max: 50 mA). One subject was excluded from further analysis because the RIII reflex threshold under influence of propofol alone already reached the maximum output of the stimulator (50 mA), after which the experimental session was discontinued.

The individual normalized RIII reflex threshold values, of responses to the noxious stimuli at the different remifentanil effect compartment concentrations are shown in FIG. 5.

The individual prediction probabilities for reactions and the absence of reactions to the noxious stimuli are shown in Table 1. While for the first drug administration protocol (fixed propofol concentrations plus variable remifentanil concentrations), no significant difference between the prediction probabilities of the RIII reflex threshold and the BIS could be observed (P>0.05, n=10, Wilcoxon signed-rank test), the prediction probabilities of the RIII reflex threshold and the BIS differed significantly (P<0.01, n=9, Wilcoxon signed-rank test) for the second drug administration protocol (high propofol concentrations plus low remifentanil concentrations vs low propofol concentrations plus high remifentanil concentrations).

For the comparison of the population prediction performance, the RIII reflex threshold values were normalized by subtraction of the first threshold that was estimated after the subject's loss of consciousness. All recorded BIS- and normalized RIII reflex values of all subjects after the individual losses of consciousness for the first drug administration protocol are shown in FIG. 3. For this drug administration protocol, the population prediction probability PK for the combined data from all subjects amounted for the BIS to 0.84 (0.02) [estimate (se)], for the normalized RIII reflex threshold to 0.86 (0.02) [estimate (se)], and for remifentanil effect compartment concentration to 0.88 (0.02) [estimate (se)]. All PK-values differed significantly from 0.5 (P<0.01, PKDMACRO), but the differences between the PK-values were not statistically significant (P>0.05, PKDMACRO).

Normalization of the BIS did not improve the prediction probability PK which amounted to 0.81 (0.03). The prediction probability for non-normalized RIII reflex threshold values amounted to 0.70 (0.04).

All recorded BIS- and normalized RIII reflex values of all subjects after the individual losses of consciousness for the second drug administration protocol are shown in FIG. 4. For this drug administration protocol, the population prediction probability PK for the combined data from all subjects amounted for the BIS to 0.64 (0.05) [estimate (se)], for the normalized RIII reflex threshold to 0.77 (0.04) [estimate (se)], and for the remifentanil effect compartment concentration to 0.67 (0.04) [estimate (se)]. These PK-values differed significantly from 0.5 (P<0.01, PKDMACRO). The PK-value of propofol effect compartment concentration amounted to 0.52 (0.03) and did not differ significantly from 0.5 (P>0.01, PKDMACRO). The difference between the PK-value of the normalized RIII reflex threshold and those of the BIS and the remifentanil effect compartment concentration was statistically significant (P<0.05, PKDMACRO). The difference between the PK-values of the BIS and the remifentanil effect compartment concentration was not statistically significant (P>0.05, PKDMACRO).

Normalization of the BIS did not improve the prediction probability PK, which amounted to 0.63 (0.05). The prediction probability for non-normalized RIII reflex threshold values amounted to 0.69 (0.04).

Table 1: Individual Prediction Probabilities for Reactions on Vocal Commands and on Noxious Stimulations

Shown are the individual prediction probability estimates P_{K} and their standard errors for reactions on vocal commands were calculated from all individual reflex recordings before the loss of reaction on the noxious stimuli. Individual prediction probabilities P_{K} for reactions on the noxious stimuli were calculated from all individual reflex recordings after the individuals' losses of consciousness. A P_{K} value of 1 stands for a 100% correct differentiation between reaction and absence of reaction, whereas a value of 0.5 represents only a 50:50 chance of a correct differentiation. (SE=Standard deviation)

| Protocol 1 | | | Protocol 2 | | |
|---|---|---|---|---|---|
| | RIII reflex | | | RIII reflex | |
| Individual | threshold | BIS | Individual | threshold | BIS |
| A: | 0.98 ± 0.02 | 0.99 ± 0.02 | L: | 0.73 ± 0.17 | 0.62 ± 0.17 |
| B: | 0.92 ± 0.07 | 0.83 ± 0.09 | M: | 0.98 ± 0.02 | 0.73 ± 0.25 |
| C: | 0.88 ± 0.08 | 0.87 ± 0.07 | N: | 0.94 ± 0.06 | 0.69 ± 0.15 |
| D: | 1.00 ± 0.00 | 0.85 ± 0.08 | O: | 0.97 ± 0.04 | 0.69 ± 0.15 |
| E: | 0.92 ± 0.06 | 0.95 ± 0.04 | P: | 0.91 ± 0.06 | 0.71 ± 0.12 |
| F: | 0.86 ± 0.08 | 1.00 ± 0.00 | Q: | 1.00 ± 0.00 | 0.33 ± 0.19 |
| G: | 0.74 ± 0.03 | 0.65 ± 0.12 | R: | 0.70 ± 0.12 | 0.54 ± 0.14 |
| H: | 0.97 ± 0.03 | 0.98 ± 0.02 | S: | 0.99 ± 0.12 | 0.58 ± 0.15 |
| I: | 0.72 ± 0.12 | 0.92 ± 0.06 | T: | 0.66 ± 0.16 | 0.63 ± 0.18 |
| K: | 1.00 ± 0.00 | 0.98 ± 0.02 | | | |
| Mean: | 0.90 | 0.90 | Mean: | 0.88 | 0.61 |
| SE: | 0.03 | 0.04 | SE: | 0.05 | 0.04 |

### REFERENCE NUMERALS

1 Apparatus (“Pain Monitor”)
2 Simulation unit
3 Measurement unit
4 Control unit
5 Signal
6 Control signal
7 Stimulation signal
8 Sedated individual
9 Calculated stimulation intensity
10 Numerical value
11 Display means

### CITED LITERATURE

Hung O R, Varvel J R, Shafer S L, Stanski D R. Thiopental pharmacodynamics. II. Quantitation of clinical and electroencephalographic depth of anesthesia. Anesthesiology 1992; 77: 237-44

Katoh T, Suzuki A, Ikeda K. Electroencephalographic derivatives as a tool for predicting the depth of sedation and anesthesia induced by sevoflurane. Anesthesiology 1998; 88: 642-50

Leslie K, Sessler D I, Smith W D, et al. Prediction of movement during propofol/nitrous oxide anesthesia. Performance of concentration, electroencephalographic, pupillary, and hemo-dynamic indicators. Anesthesiology 1996; 84: 52-63

Minto C F, Schnider T W, Egan T D, et al. Influence of age and gender on the pharmacokinetics and pharmacodynamics of remifentanil.l. Model development. Anesthesiology 1997; 86: 10-23

Petersen-Felix S, Arendt-Nielsen L, Bak P, Roth D, Fischer M, Bjerring P, Zbinden A M. Analgesic effect in humans of subanaesthetic isoflurane concentrations evaluated by experimentally induced pain. Br J Anaesth. 1995 July; 75:55-60.

Petersen-Felix S, Arendt-Nielsen, Bak, Fischer, Bjerring, Zbinden A M. The effects of isoflurane on repeated nociceptive stimuli (central temporal summation). Pain. 1996 February; 64:277-81.

Rehberg B, Grunewald M, Baars J, Fuegener K, Urban B W, Kox W J. Monitoring of immobility to noxious stimulation during sevoflurane anesthesia using the spinal H-reflex. Anesthesiology 2004; 100: 44-50

Rhudy J L, France C R. Defining the nociceptive flexion reflex (NFR) threshold in human participants: a comparison of different scoring criteria. Pain 2007; 128: 244-53

Sandrini G, Serrao M, Rossi P, Romaniello A, Cruccu G, Willer J C: The lower limb flexion reflex in humans. Prog. Neurobiol. 2005; 77: 353-95

Schnider T W, Minto C F, Gambus P L, et al. The influence of method of administration and covariates on the pharmacokinetics of propofol in adult volunteers. Anesthesiology 1998; 88: 1170-82

Shafer S L, Stanski D R: Defining Depth of Anesthesia, Modern Anesthetics, 1 edition. Edited by Schüttler J, Schwilden H. Berlin, Heidelberg, Springer-Verlag, 2008, 409-23

Skljarevski V, Ramadan N M: The nociceptive flexion reflex in humans—review article. Pain 2002; 96: 3-8

Smith W D, Dutton R C, Smith N T. Measuring the performance of anesthetic depth indicators. Anesthesiology 1996; 84: 38-51

von Dincklage F, Hackbarth M, Schneider M, Baars J H, Rehberg B: Introduction of a continual RIII reflex threshold tracking algorithm. Brain Res. 2009; 1260: 24-29

Willer J C: Studies on pain. Effects of morphine on a spinal nociceptive flexion reflex and related pain sensation in man. Brain Res. 1985; 331: 105-14

Willer J C, Bergeret S, Gaudy J H: Epidural morphine strongly depresses nociceptive flexion reflexes in patients with postoperative pain. Anesthesiology 1985; 63: 675-80

1-9. (canceled)

10. A method for determining the level of analgesia of a sedated or anesthetized individual comprising the steps of:
providing an apparatus, wherein the apparatus comprises:
a stimulation unit for generating a stimulation signal for pain-specific reflex,
a measurement unit for recording a pain-specific reflex signal in response to the stimulation signal, and
a control unit for adapting the intensity of the stimulation signal, and wherein
the control unit decreases the intensity of the stimulation signal when the pain-specific reflex signal that is caused by the stimulation signal is above an index value of the reflex response, and
the control unit increases the intensity of the stimulation signal when the pain specific reflex response caused by the stimulation signal is below a predetermined index value of the reflex response,
generating a stimulation signal for pain-specific reflex with the stimulation unit, recording a pain-specific reflex signal in response to the stimulation signal with the measurement unit, and
adapting the intensity of the stimulation signal with the control unit, and
determining the level of analgesia using the recorded pain-specific reflex signal.

11. The method of claim 10, comprising the step of analyzing the recorded reflex response with the control unit or determining artefacts with the control unit.

12. A method for determining a level of analgesia of a sedated individual, comprising the following steps:
generating a reflex stimulation signal for deploying a pain-specific reflex response,
adapting the intensity of the reflex stimulation signal based on a feedback loop,
determining a calculated stimulation signal intensity,
triggering a reflex response signal of a predetermined index value, and
determining the level of analgesia of the sedated individual using the calculated stimulation signal intensity.

13. The method according to claim 12, wherein the reflex stimulation signal is an electrical, mechanical, or thermal signal.

14. The method according to claim 12, additionally comprising the step of recording the reflex response signal in response to the reflex stimulation signal.

15. The method of claim 12 wherein the pain-specific reflex is a nociceptive reflex.

16. The method of claim 15 wherein the nociceptive reflex is an eye blink reflex or a flexor reflex.

17. The method of claim 16 wherein the flexor reflex is a flexor reflex of a lower extremity.

18. The method of claim 12 wherein the sedated individual is sedated with a sedative that is selected from the group consisting of propofol, benzodiazepines and alpha-2-antagonists.

19. The method of claim 12 wherein the analgesia is performed using an analgetic drug that is selected the group consisting of opioid analgesics and non-opioid analgesics.

20. The method of claim 12 wherein the reflex stimulation signal is an impulse or a sequence of impulses of similar amplitude.

21. The method of claim 12 wherein the reflex response signal is registered within a time frame of 1 ms to 1 s after generating the stimulation signal.

22. The method of claim 21 further comprising, after the step of triggering the reflex response signal of the predetermined value, the step of:
adapting a current of a subsequent reflex stimulation signal depending on the magnitude of the registered reflex response signal, wherein the step of adapting the current of the subsequent reflex stimulation signal comprises:
increasing the current of the subsequent reflex stimulation signal when a predefined index value of the reflex response is not reached, or
decreasing the current of the subsequent reflex stimulation signal when the predefined index value of the reflex response is exceeded,
and further comprising the step of:
determining a calculated stimulation intensity for triggering the reflex response using the amplitude of the predetermined index value.

23. The method of claim 12 wherein the calculated stimulation signal intensity is a parameter of a level of:
analgesia of the sedated individual, or
pain sensitivity of the sedated individual.
